(19) United States
(12) Patent Application Publication
(10) Pub. No.: US 2018/0214600 A1
(43) Pub. Date: Aug. 2, 2018

(54) TEXTURED ARTICLES FOR ENHANCED CELL FORMATION AND METHODS OF MAKING THEREOF

(71) Applicants: UNIVERSITY OF FLORIDA RESEARCH FOUNDATION, INC., Gainesville, FL (US); SHARKLET TECHNOLOGIES, INC., Aurora, CO (US)

(72) Inventors: Chelsea Marie MAGIN, Denver, CO (US); Anthony B. BRENNAN, Gainesville, FL (US)

(21) Appl. No.: 15/748,302

(22) PCT Filed: Jul. 29, 2016
(86) PCT No.: PCT/US2016/044750

§ 371 Date: Jan. 29, 2018

Related U.S. Application Data
(60) Provisional application No. 62/198,897, filed on Jul. 30, 2015.

(51) Int. Cl. A61L 27/18; A61L 27/38; A61L 27/54; A61L 27/58

(52) U.S. Cl. CPC A61L 27/18; A61L 27/3834; A61L 2400/18; A61L 27/58; A61L 2430/38; A61L 27/54

(57) ABSTRACT

Disclosed herein is an article including a substrate having at least one surface that has a texture and a first type of growing cells, wherein an average length of features that form the texture is operative to facilitate cell growth, cell orientation, cell morphology and cell differentiation. Disclosed herein too is a method including forming a first layer of a polymeric material on at least one surface of a substrate; texturing the first layer to form a textured first layer; and forming a first type of growing cells on the textured first layer, wherein an average length of features that form the textured first layer is operative to facilitate cell growth, cell orientation, cell morphology and cell differentiation.

### BACKGROUND

This disclosure relates to textured articles for enhanced cell formation.

Medical treatments such as spinal fusion, provide a corrective surgery which utilizes an autologous bone grafting technique to treat and repair bone injuries in patients. Over 400,000 spinal fusion procedures are performed in the United States each year. Autologous bone grafting is a procedure in which bone is taken from the patient and transferred to the portion of the same patient's spine to be fused. However, this approach is limited in that standard spinal fusion procedures result in complications in up to 50% of patients and up to 35% nonunion rates.

Engineering bone tissue grown from stem cells provides an alternative to this procedure. Human Mesenchymal Stem Cells (HMSC) are cells which are capable of replicating as undifferentiated cells or differentiating into a tissue-specific cell. More specifically, HMSCs are capable of differentiating into bone marrow, bone, cartilage, tendon, muscle, or fat cells. However, standard procedures including using chemical additives to induce differentiation suffer from drawbacks with regard to adequately controlling variability, which leads to heterogeneous cell populations, i.e., resulting in a mix of cells that includes, but is not limited to, those with the desired cell function (or tissue-specific cell), e.g., bone. These artificial chemical treatments do not induce differentiation consistently, which may be detrimental to both experimental and clinical outcomes.

Therefore, a need exists for an article and method which enhances cell formation by more precisely controlling the differentiation of human stem cells into the desired tissue-specific cells, e.g., bone, and which also reduces nonunion rates in spinal fusion procedures without the addition of chemical additives.

### SUMMARY

Disclosed herein is an article comprising a substrate having at least one surface that has a texture; and a first type of growing cells, wherein an average length of features that form the texture is operative to facilitate cell growth, cell orientation, cell morphology and cell differentiation.

Disclosed herein too is a method comprising forming a first layer of a polymeric material on at least one surface of a substrate texturing the first layer to form a textured first layer; and comprising forming a first type of growing cells on the textured first layer, wherein an average length of features that form the textured first layer is operative to facilitate cell growth, cell orientation, cell morphology and cell differentiation.

### BRIEF DESCRIPTION OF THE FIGURES

FIG. 1 is an illustration of the cellular aspect ratio and orientation of a cell disposed on a Sharklet™ (SK) surface;

FIG. 2(A) shows fluorescent microscopic images of human mesenchymal stem cells on SM (smooth) surfaces;

FIG. 2(B) shows fluorescent microscopic images of human mesenchymal stem cells on −1.7SK2×2 Sharklet textured surfaces;

FIG. 2(C) shows fluorescent microscopic images of human mesenchymal stem cells on +1.7SK2×2 Sharklet textured surfaces;

FIG. 2(D) shows fluorescent microscopic images of human mesenchymal stem cells on −1.5SK10×5 SK surfaces;

FIG. 2(E) shows fluorescent microscopic images of human mesenchymal stem cells on +1.5SK10×5 SK textured surfaces;

FIG. 3 is a graph of the average cellular aspect ratio of the cells formed on the smooth and patterned SK surfaces of FIG. 2;

FIG. 4(A) is a photograph that shows hMSC cultured in growth media on a SM (smooth) surface;

FIG. 4(A) is a photograph that shows hMSC cultured in growth media on a SK (Sharklet) textured surface;

FIG. 4(C) is a photograph that shows hMSC cultured on a SK (Sharklet) textured surface in growth media stained for alkaline phosphatase (ALP) as an early marker for osteogenesis;

FIG. 5 is a graph of the ALP score for hMSCs cultured on the smooth and patterned SK surfaces of FIGS. 4(A), 4(B) and 4(C) respectively;

FIG. 6(A) is a photograph that shows hMSC cultured in osteogenic media on a SM (smooth) surface;

FIG. 6(B) is a photograph that shows hMSC cultured in osteogenic media on a SK (Sharklet) surface;

FIG. 6(C) is a photograph that shows hMSC cultured in osteogenic media on a SK (Sharklet) surface stained for alkaline phosphatase (ALP) as an early marker for osteogenesis;

FIG. 7 is a graph of the ALP score for hMSCs cultured on the smooth and patterned SK surfaces of 6(A), 6(B) and 6(C) respectively;

FIG. 8(A) shows hMSCs cultured in osteogenic media on a SM (smooth) surface;

FIG. 8(B) shows hMSCs cultured in osteogenic media on a Sharklet (SK) textured surface and stained for Alizarin red to detect calcium production as a functional output of bone cells;

FIG. 9 is a graph of the concentration of Alizarin red staining of hMSCs cultured on SM and SK surfaces, based on Alizarin red extraction and quantification via absorbance where Alizarin red selectively labels calcium produced by the cells;

FIG. 10(A) is a photograph that shows the influence of Sharklet textured micropatterns on hMSC migration on Sharklet patterns having the +1.7SK2×2 Sharklet texture;

FIG. 10(B) is a photograph that shows the influence of Sharklet textured micropatterns on hMSC migration on Sharklet patterns having the +1.5SK10×5Sharklet texture;

FIG. 10(C) is a photograph that shows hMSC migration on smooth (SM) surfaces;

FIG. 10(D) is a bar graph that reflects normalization coverage on smooth (SM) surfaces and textured surfaces having the +1.7SK2×2 and +1.5SK10×5 texture;

FIG. 11(A) shows photomicrographs that show growing cells on smooth and textured surfaces. These figures demonstrate that Sharklet micropatterns enhance MSC significantly compared to SM surfaces and suggest that micropatterns can be optimized to promote MSC migration onto spinal fusion devices;

FIG. 11(B) is a bar graph that shows the ALP score variation with time on smooth and textured surfaces; and

FIG. 11(C) is a bar graph that shows the Alizaren Red Concentration variation with time.

### DETAILED DESCRIPTION

Disclosed herein is a patterned article that enhances cell growth, cell orientation, cell morphology and cell differentiation. The article comprises a substrate having at least one surface that has a texture and a first type of growing cells. The textured surface comprises a plurality of features. The average length of the features that form the texture influences the cell morphology and orientation and consequently, the resulting tissue-specific cell function, e.g., bone tissue.

The substrate may be formed of any material that is suitable for facilitating cell growth. Non-limiting examples include polystyrene, polytetrafluoroethylene or polyorganosiloxane. The substrate has a texture disposed on at least one surface. In an embodiment, the substrate is a tissue culture dish. The texturing facilitates cell growth along particular directions determined by the orientation of the texture. The texturing also facilitates cell differentiation of stem cells.

In an embodiment, the article is vessel for growing cell cultures. In another embodiment, the article is a grafting scaffold used to generate grafts from cell cultures, e.g., a bone grafting scaffold to generate a bone graft.

In an embodiment, the textured surface and/or the substrate comprise a thermoplastic polymer. In another embodiment, the textured surface and/or substrate comprises any material that is suitable for use in bone grafts. Non-limiting examples include polystyrene, polytetrafluoroethylene, polyorganosiloxane, calcium phosphate, polyether ether ketone (PEEK), bioactive glass, a ceramic material, a composite of a ceramic material and polymers, a composite of bioactive glass and a polymer, or the like, or a combination thereof.

In an exemplary embodiment, polydimethylsiloxane is a preferred polymer for the textured surface. The texture may be formed on at least one surface of the substrate using any suitable technique, including but not limited to, injection molding, hot embossing, casting, laser etching, chemical etching, or the like.

The surface of the substrate may have any kind of texture. Examples of surface textures are detailed in US 2005/0003146 A1 to Spath, U.S. Pat. No. 7,143,709 B2 to Brennan et al., and U.S. patent application Ser. No. 12/550,870 to Brennan et al., the entire contents of which are hereby incorporated by reference in their entirety.

In an embodiment, the textured surface comprises a plurality of spaced apart features; the features arranged in a plurality of groupings; the groupings of features being arranged with respect to one another so as to define a tortuous pathway when viewed in a first direction. When viewed in a second direction, the groupings of features are arranged to define a linear pathway. Radial patterns may also be used. In radial patterns the patterns emanate from a focal point and spread outwards in a radial direction.

In another embodiment, when viewed in a second direction, the pathway between the features may be non-linear and non-sinusoidal. In other words, the pathway can be non-linear and aperiodic. In yet another embodiment, the pathway between the features may be linear but of a varying thickness. The plurality of spaced apart features may be projected outwards from a substrate surface or projected into the substrate surface. In one embodiment, the plurality of spaced apart features have the same chemical composition as the substrate. In another embodiment, the plurality of spaced apart features has a chemical composition that is different than chemical composition of the substrate.

The plurality of spaced apart features each have at least one microscale (micrometer or nanometer sized) dimension and has at least one neighboring feature having a substantially different geometry. The average first feature spacing between the adjacent features is between about 10 nanometers to about 100 micrometers in at least a portion of the textured surface, wherein said plurality of spaced apart features are represented by a periodic function. In one embodiment, the first feature spacing is between about 0.5 micrometers (μm) and about 5 μm in at least a portion of the textured surface. In another embodiment, the first feature spacing is between about 15 and about 60 μm in at least a portion of the textured surface. As noted above, the periodic function comprises two different sinusoidal waves. In one embodiment, the topography resembles the topography of shark-skin (e.g., a Sharklet). In another embodiment, the pattern comprises at least one multi-element plateau layer disposed on a portion of the substrate, wherein a spacing distance between elements of the plateau layer provides a second feature spacing; the second feature spacing being substantially different when compared to said first feature spacing. It is to be noted that each of the features of the plurality of features are separated from each other and do not contact one another.

The pattern of the texture is separated from a neighboring pattern by a tortuous pathway. The tortuous pathway may be represented by a periodic function. The periodic functions may be different for each tortuous pathway. In one embodiment, the patterns can be separated from one another by tortuous pathways that can be represented by two or more periodic functions. The periodic functions may comprise a sinusoidal wave. In an exemplary embodiment, the periodic function may comprise two or more sinusoidal waves.

In another embodiment, when a plurality of different tortuous pathways are represented by a plurality of periodic functions respectively, the respective periodic functions may be separated by a fixed phase difference. In yet another embodiment, when a plurality of different tortuous pathways are represented by a plurality of periodic functions respectively, the respective periodic functions may be separated by a variable phase difference.

In one embodiment, the plurality of spaced apart features have a substantially planar top surface. In another embodiment, a multi-element plateau layer can be disposed on a portion of the surface, wherein a spacing distance between elements of said plateau layer provide a second feature spacing; the second feature spacing being substantially different when compared to the first feature spacing.

In one embodiment, a sum of a number of features shared by two neighboring groupings is equal to an odd number. In another embodiment, a sum of a number of features shared by two neighboring groupings is equal to an even number. Details of the texture (in the form of Figures may be seen in U.S. patent application Ser. No. 12/550,870 to Brennan et al., the entire contents of which are hereby incorporated by reference in their entirety.

A first type of growing cells is disposed on the textured surface. In an embodiment, the first type of growing cells are selected from the group consisting of embryonic stem cells, adult stem cells and induced pluripotent stem cells. In an exemplary embodiment, the first type of growing cells are human mesenchymal stem cells (hMSC).

In the mesengenic process of the proliferation, commitment, lineage progression, differentiation and maturation of human mesenchymal stem cells, the cell morphology of mesenchymal stem cells change accordingly, after proliferating via marrow stroma, osteogenesis, chondrogenesis, tendogenesis, myogenesis or adipogenesis pathways into bone marrow, bone, cartilage, tendon, muscle and fat tissue. The cell morphology (or shape) of the cell during cell growth during the mesengenic process determines the resultant cell function. The textured surface influences cell shape and directs cell differentiation into a particular type of cell function, e.g., bone tissue.

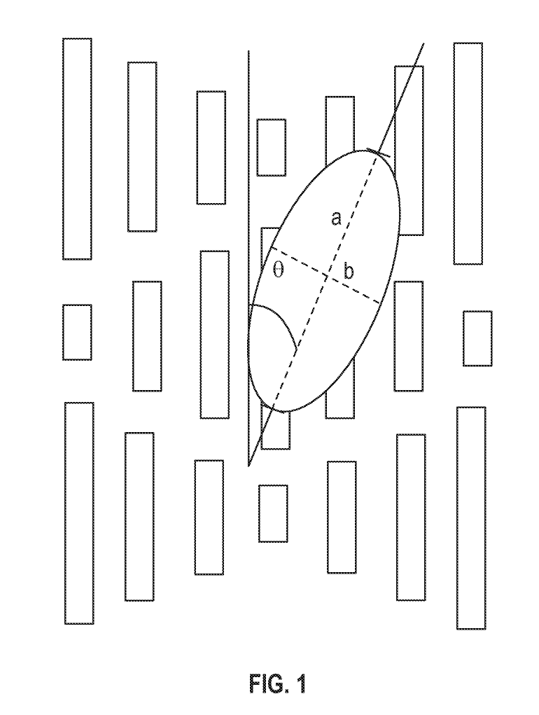

Referring to FIG. 1, an illustration of the cellular aspect ratio and orientation of a cell disposed on a Sharklet™ (SK) surface is shown. As shown in FIG. 1, cell elongation is quantified by calculating the average cellular aspect ratio (AR) using the formula AR=a/b where a is the length of the long axis of the cell and b is the length of the short axis of the cell. The orientation (0) of the cell is determined by measuring the angle between the long axis of the cell and the direction of the texture features.

In an embodiment, the average length of features that form the texture is operative to facilitate cell differentiation of the first type of growing cells into a particularly selected tissue-specific cell such as bone tissue. In an exemplary embodiment, as the average length of the texture features is increased, the average cellular aspect ratio will also increase. In another embodiment, the average cellular aspect ratio of the first type of growing cells is increased compared to a substrate that does not have the texture.

The cells interact with a surface of a substrate through focal adhesions, which are mechano-sensitive signaling complexes that grow and adapt in response to topographically modified substrates, resulting in intracellular tension and cellular anisotropy. The discontinuous features in Sharklet microtopographies induce high levels of cellular anisotropy and allow for focal adhesions to be precisely guided for a greater level of control over the morphology of a cell population, and consequently over cell differentiation. In an embodiment, the SK surface exhibits higher levels of cellular anisotropy than a smooth surface or a surface which includes an elongated channel or pillar pattern.

Disclosed herein is also a method comprising forming a first layer of a polymeric material on at least one surface of a substrate; texturing the first layer to form a textured first layer; and forming a first type of growing cells on the textured first layer, wherein an average length of features that form the textured first layer is operative to facilitate cell growth, cell orientation, cell morphology and cell differentiation.

The textured surface enhances tissue cell formation by using the textured pattern to control the cell morphology and orientation of cells, thereby targeting tissue-specific cellular functions of stem cells, e.g., bone tissue. In an exemplary embodiment, the resulting bone tissue exhibits a reduction in nonunion rates in comparison to bone tissue which is not generated using the textured surface.

### EXAMPLES

### Example 1

Smooth (SM) and micropatterned SK samples were fabricated by casting polydimethylsiloxane elastomer (Xiameter RTV-4232-T2, Dow Corning; PDMSe) against negative silicon wafer molds. Human Mesenchymal Stem Cells (hMSC) were cultured on these wafer molds for three days in growth media, and then stained, fixed and measured.

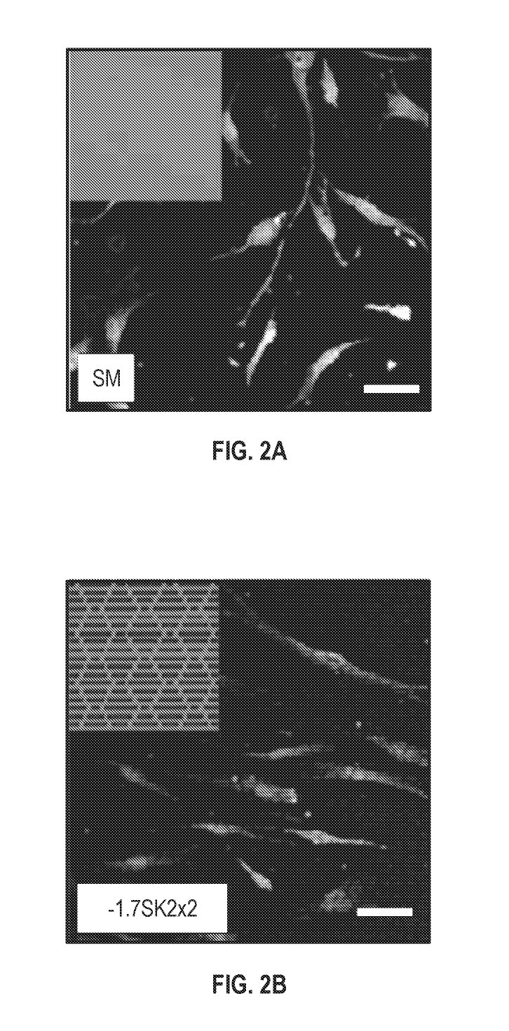

Fluorescent microscopy images were taken of each sample and the average cellular aspect ratio for each sample was calculated. FIG. 2 shows fluorescent microscopic images of human mesenchymal stem cells on SM (smooth) and −1.7SK2×2, +1.7SK2×2, −1.5SK10×5 and +1.5SK10×5 SK surfaces. FIG. 2(A) shows fluorescent microscopic images of human mesenchymal stem cells on SM (smooth) surfaces. FIG. 2(B) shows fluorescent microscopic images of human mesenchymal stem cells on −1.7SK2×2 Sharklet textured surfaces. FIG. 2(C) shows fluorescent microscopic images of human mesenchymal stem cells on +1.7SK2×2 Sharklet textured surfaces. FIG. 2(D) shows fluorescent microscopic images of human mesenchymal stem cells on −1.5SK10×5 SK surfaces. FIG. 2(E) shows fluorescent microscopic images of human mesenchymal stem cells on +1.5SK10×5 SK textured surfaces.

The insets show confocal micrographs of the underlying SM and patterned SK surfaces. The average length of the rectangular features of the SK surface varied from 4 to 80 micrometers.

The nomenclature adopted here (e.g., +1.7SK2×2) should deciphered as follows: The +1.7 indicates the height of the texture above the base surface while the SK refers to a Sharklet pattern depicted and described in U.S. Pat. No. 7,143,709 B2 to Brennan et al., and Patent Application having Ser. No. 12/550,870 to Brennan et al. A negative sign (−) preceding the 1.7 would indicate that the texture is below the base surface. The first 2 in SK2×2 stands for the width of each feature in the pattern while the second 2 stands for the spacing between the features in the pattern.

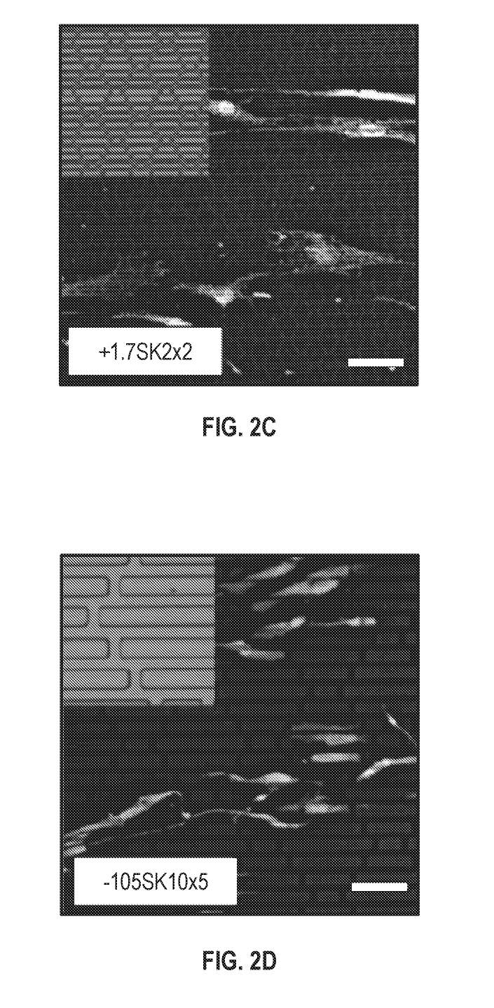

The average cellular aspect ratio (AR) was calculated using the formula AR=a/b where a is the length of the long axis of the cell and b is the length of the short axis of the cell, as shown in FIG. 1. The average cellular aspect ratios of the cells formed on the smooth and patterned SK are plotted in the graph in FIG. 3. These results show that significantly higher average cellular aspect ratios are obtained using the SK surface in comparison to the SM surface. These results also show that SK micropatterns that as the average feature length increases, the average cellular aspect ratio also increases. Thus, the SK surfaces can be tailored to modulate cell morphology, and to thereby control cell differentiation.

### Example 2

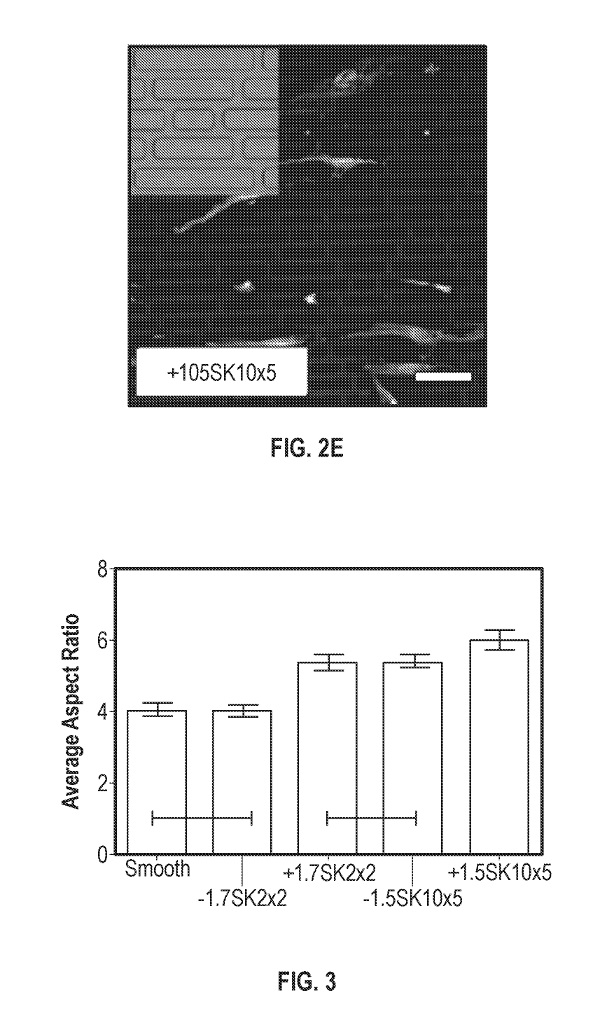

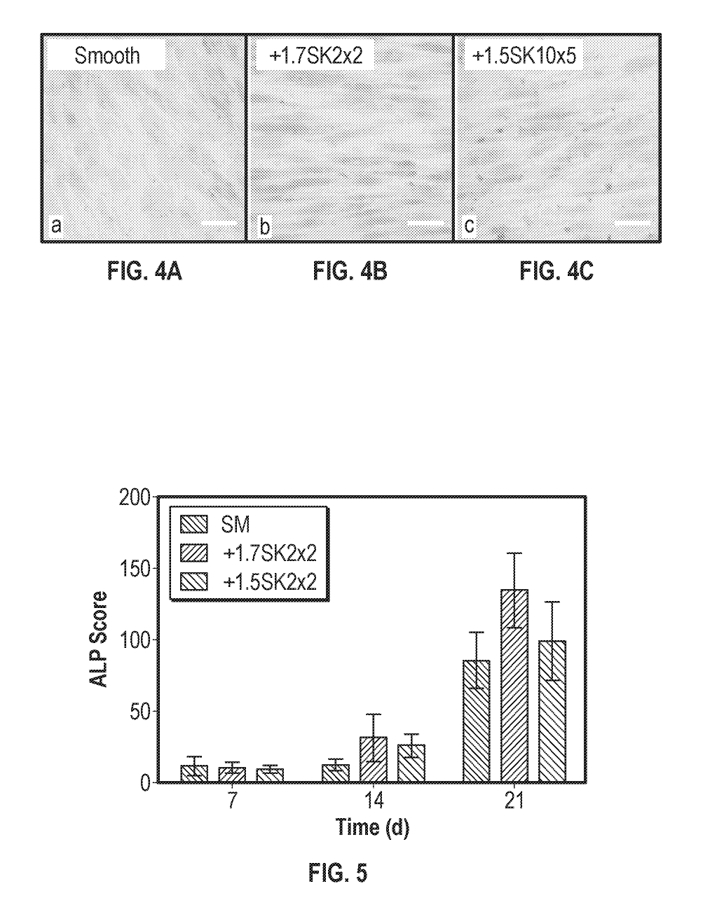

FIGS. 4(A), 4(B) and 4(C) show hMSC cultured in growth media on SM (smooth) and SK (Sharklet) surfaces and stained for alkaline phosphatase (ALP) as an early marker for osteogenesis after 7, 14 and 21 days. FIG. 5 is a graph of the ALP score for the hMSCs cultured on the smooth and patterned SK surfaces of 4(A), 4(B) and 4(C) where the scale bars on the y-axis appear in 50 μm increments. Staining reveals that the ALP production is significantly increased when the SK surfaces are employed in comparison to the SM surface. These results show that the SK surfaces induce osteogenic and bone cell differentiation in hMSCs.

### Example 3

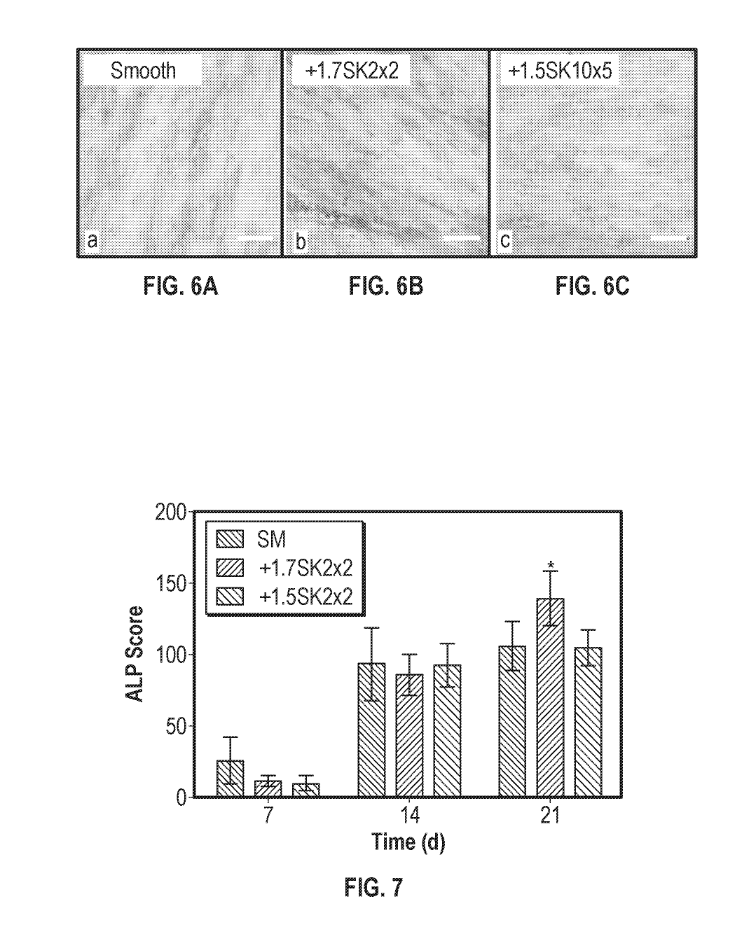

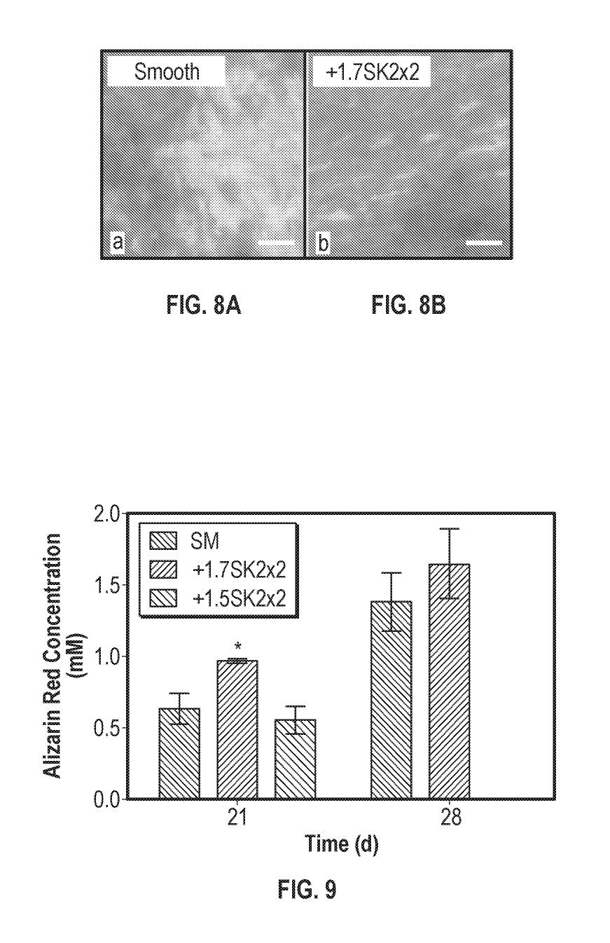

FIGS. 6(A), 6(B) and 6(C) show hMSC cultured in osteogenic media on SM (smooth) and SK (Sharklet) surfaces, stained for alkaline phosphatase (ALP) as an early marker for osteogenesis. FIG. 7 is a graph of the ALP score for the hMSCs cultured on the smooth and patterned SK surfaces of 6(A), 6(B) and 6(C) respectively. As may be seen from the graph in FIG. 7, increased ALP production is observed with the +1.7SK2×2 SK surface in comparison to the +1.5SK2×2 and the SM surface.

### Example 4

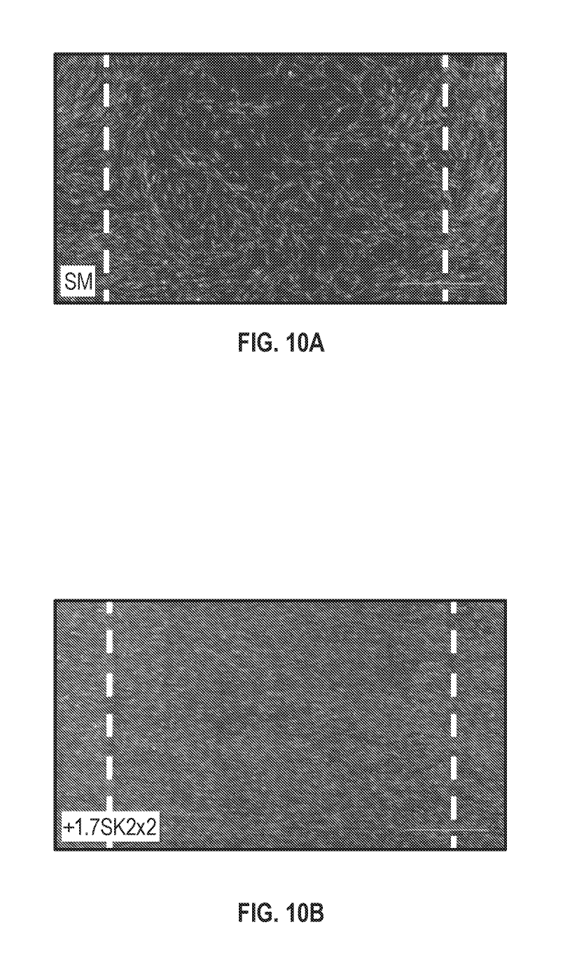

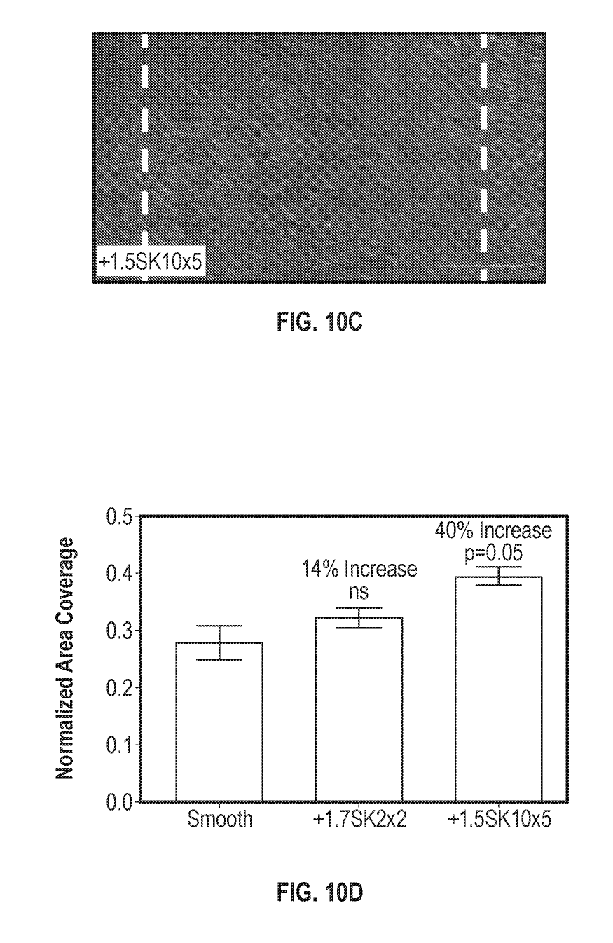

FIGS. 8(A) and 8(B) show hMSC cultured in osteogenic media on SM (smooth) and SK (Sharklet) surfaces, stained for Alizarin red to detect calcium production as a functional output of bone cells. FIG. 9 is a graph of the concentration of Alizarin red staining of hMSCs cultured on SM tissue culture polystyrene (TCPS) and SK surfaces, based on Alizarin red extraction and quantification via absorbance where Alizarin red selectively labels calcium produced by the cells. As may be seen from the graph in FIG. 9, increased calcium production is observed with the +1.7SK2×2 SK surface in comparison to the +1.5SK2×2 and the SM surface.

### Example 5

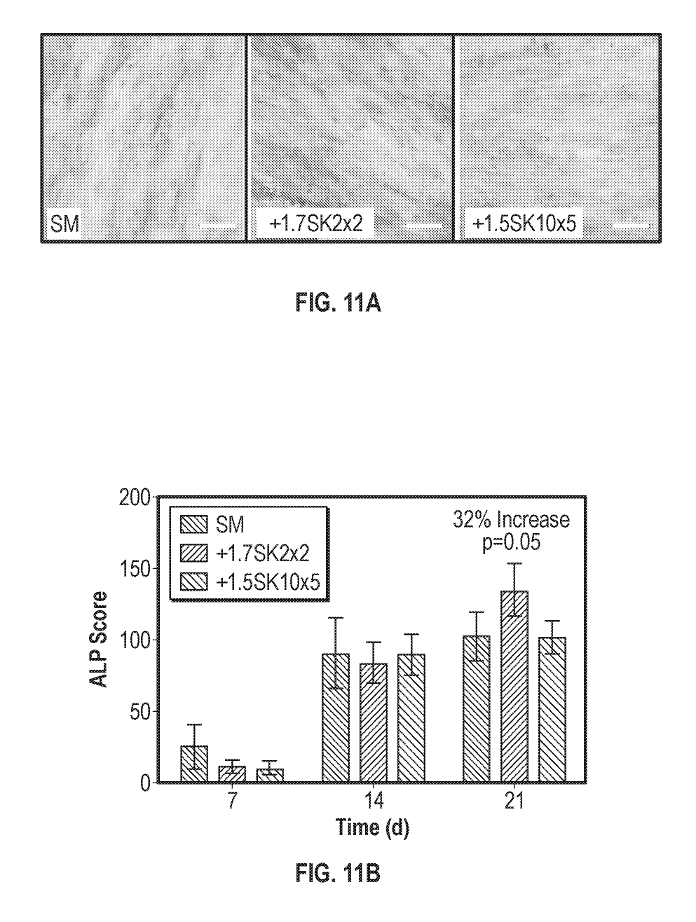

FIGS. 10A-10D show the influence of Sharklet micropatterns on hMSC migration, two micropatterns (+1.7SK2×2 and +1.5SK10×5) were replicated in polydimethylsiloxane elastomer (PDMSe) and tested in a modified scratch wound assay compared to smooth (SM) controls. FIG. 10A is a photograph that shows the influence of Sharklet textured micropatterns on hMSC migration on Sharklet patterns having the +1.7SK2×2 Sharklet texture. FIG. 10A is a photograph that shows the influence of Sharklet textured micropatterns on hMSC migration on Sharklet patterns having the +1.5SK10×5Sharklet texture. FIG. 10C is a photograph that shows hMSC migration on smooth (SM) surfaces. FIG. 10D is a bar graph that reflects normalization coverage on smooth (SM) surfaces and textured surfaces having the +1.7SK2×2 and +1.5SK10×5 texture.

Representative fluorescent microscopy images of MSCs on SM and Sharklet micropatterns are depicted here where the dashed white lines represent the wounded area. MSCs were cultured on these samples in the migration assay format for 7 days in growth media, fixed, stained with a CellTracker membrane dye and the area covered by cells within the artificial wound was measured and compared.

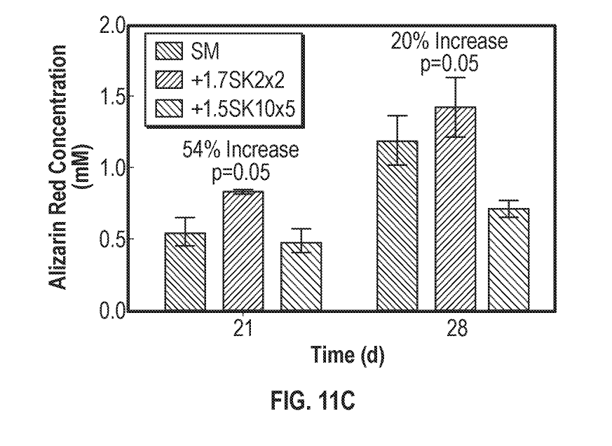

Results in FIG. 11 demonstrate that Sharklet micropatterns enhance MSC significantly compared to SM surfaces and suggest that micropatterns can be optimized to promote MSC migration onto spinal fusion devices. Scale bar, 1 mm.

FIG. 11(A) shows photomicrographs that show growing cells on smooth and textured surfaces. These figures demonstrate that Sharklet micropatterns enhance MSC significantly compared to SM surfaces and suggest that micropatterns can be optimized to promote MSC migration onto spinal fusion devices. FIG. 11(B) is a bar graph that shows the ALP score variation with time on smooth and textured surfaces and FIG. 11(C) is a bar graph that shows the Alizaren Red Concentration variation with time.

In summary, a textured article may be used for growing cells. The growing cells are disposed on the texture. The average length of features that form the texture is operative to facilitate cell growth, cell orientation, cell morphology and cell differentiation. The texture comprises a plurality of spaced features; wherein each feature has a substantially different geometry than a neighboring feature; the plurality of spaced features arranged in a plurality of groupings, the spaced features within each of the groupings being spaced apart at an average distance of about 10 nanometers to about 200 micrometers; the adjacent groupings of features being spaced from each other to define an intermediate tortuous pathway.

In an embodiment, the average cellular aspect ratio of the growing cells is increased when compared with cells that are grown on a substrate that does not have at least one surface that has the texture.

The growing cells are selected from the group consisting of embryonic stem cells, adult stem cells and induced pluripotent stem cells. In an embodiment, the growing cells are human mesenchymal stem cells.

In an embodiment, the average size and arrangement of features that form the texture is operative to facilitate cell differentiation of the first type of growing cells into human bone cells. In another embodiment, the average size and arrangement of features that form the texture is operative increase cell migration compared to a surface without the texture.

As detailed above, the substrate upon which the growing cells are disposed is textured. In an embodiment, the texture comprises polystyrene, polytetrafluoroethylene, polyorganosiloxane, calcium phosphate, polyether ether ketone, bioactive glass, a ceramic material, a composite of a ceramic material and polymers, a composite of bioactive glass and a polymer, or a combination thereof. In another embodiment, the substrate comprises polystyrene, polytetrafluoroethylene, polyorganosiloxane, calcium phosphate, polyether ether ketone, bioactive glass, a ceramic material, a composite of a ceramic material and polymers, a composite of bioactive glass and a polymer, or a combination thereof.

In yet another embodiment, the texture comprises a biodegradable polymer. The biodegradable polymer is polylactic-glycolic acid, copolymers of polyurethane and polylactic-glycolic acid, poly-caprolactone, copolymers of polylactic-glycolic acid and poly-caprolactone, polyhydroxy-butyrate-valerate (PHBV), polyorthoester (POE), polyethylene oxide-butylene terephthalate (PEO-PBTP), poly-D,L-lactic acid-p-dioxanone-polyethylene glycol block copolymer (PLA-DX-PEG), or a combination comprising at least one of the foregoing biodegradable polymers.

It is to be understood that while the invention has been described in conjunction with the preferred specific embodiments thereof, that the foregoing description as well as the examples, which follow are intended to illustrate and not limit the scope of the invention. Other aspects, advantages and modifications within the scope of the invention will be apparent to those skilled in the art to which the invention pertains.

1. An article comprising:
a substrate having at least one surface that has a texture; and
a first type of growing cells, wherein an average length of features that form the texture is operative to facilitate cell growth, cell orientation, cell morphology, and cell differentiation.

2. The article of claim 1, wherein the first type of growing cells are disposed on the texture.

3. The article of claim 1, wherein the texture comprises a plurality of spaced features; wherein each feature has a substantially different geometry than a neighboring feature; wherein the plurality of spaced features are arranged in a plurality of groupings, the spaced features within each of the groupings being spaced apart at an average distance of about 10 nanometers to about 200 micrometers; and wherein adjacent groupings of features are spaced from each other to define an intermediate tortuous pathway.

4. The article of claim 1, wherein an average cellular aspect ratio of the first type of growing cells is increased compared with cells grown on a non-textured substrate.

5. The article of claim 1, wherein the first type of growing cells are selected from the group consisting of embryonic stem cells, adult stem cells, and induced pluripotent stem cells.

6. The article of claim 1, wherein the first type of growing cells are human mesenchymal stem cells.

7. The article of claim 1, wherein an average size and arrangement of features that form the texture is operative to facilitate cell differentiation of the first type of growing cells into human bone cells.

8. The article of claim 1, wherein an average size and arrangement of features that form the texture is operative to increase cell migration compared to a surface without the texture.

9. The article of claim 1, wherein the texture comprises polystyrene, polytetrafluoroethylene, polyorganosiloxane, calcium phosphate, polyether ether ketone, bioactive glass, a ceramic material, a composite of a ceramic material and polymers, a composite of bioactive glass and a polymer, or a combination thereof.

10. The article of claim 1, wherein the texture comprises a biodegradable polymer, and wherein the biodegradable polymer is polylactic-glycolic acid, copolymers of polyurethane and polylactic-glycolic acid, poly-caprolactone, copolymers of polylactic-glycolic acid and poly-caprolactone, polyhydroxy-butyrate-valerate (PHBV), polyorthoester (POE), polyethylene oxide-butylene terephthalate (PEO-PBTP), poly-D,L-lactic acid-p-dioxanone-polyethylene glycol block copolymer (PLA-DX-PEG), or a combination comprising at least one of the foregoing biodegradable polymers.

11. (canceled)

12. (canceled)

13. A method comprising:
forming a first layer of a polymeric material on at least one surface of a substrate;
texturing the first layer to form a textured first layer; and
forming a first type of growing cells on the textured first layer, wherein an average length of features that form the textured first layer is operative to facilitate cell growth, cell orientation, cell morphology and cell differentiation.

14. The method of claim 13, wherein the first type of growing cells are disposed on the textured first layer.

15. The method of claim 13, wherein the textured first layer comprises a plurality of spaced features; wherein each feature has a substantially different geometry than a neighboring feature; wherein the plurality of spaced features are arranged in a plurality of groupings, the spaced features within each of the groupings being spaced apart at an average distance of about 10 nanometers to about 200 micrometers; and wherein adjacent groupings of features are spaced from each other to define an intermediate tortuous pathway.

16. The method of claim 13, wherein an average cellular aspect ratio of the first type of growing cells is increased compared to a substrate that does not have at least one surface that has the texture.

17. (canceled)

18. The method of claim 13, wherein the first type of growing cells are human mesenchymal stem cells.

19. (canceled)

20. (canceled)
